(19) United States
(12) Patent Application Publication
(10) Pub. No.: US 2017/0151148 A1
(43) Pub. Date: Jun. 1, 2017

(54) NAIL GEL POLISH AND ITS MANUFACTURING METHOD

(71) Applicant: Shanghai O'Nine Technologies Ltd., Shanghai (CN)

(72) Inventors: Laipeng Chen, Shanghai (CN); Mingtai Ge, Shanghai (CN); Yueyun Dong, Shanghai (CN)

(21) Appl. No.: 15/215,555

(22) Filed: Jul. 20, 2016

(30) Foreign Application Priority Data
Dec. 1, 2015 (CN) 201510867377.1

(51) Int. Cl. A61K 8/49; A61K 8/87; A61K 8/86; A61K 8/35; A61K 8/55; A61K 8/26; A61Q 3/02; A61K 8/36

(52) U.S. Cl. CPC A61K 8/4986; A61Q 3/02; A61K 8/87; A61K 8/36; A61K 8/35; A61K 8/55; A61K 8/26; A61K 8/86; A61K 2800/95; A61K 2800/43; A61K 2800/48; A61K 2800/412; A61K 2800/805; A61K 2800/54; A61K 2800/5922

(57) ABSTRACT

This invention relates to the field of daily chemical products. Disclosed is a nail gel polish and its manufacturing method. The nail coatings used in human finger mainly are nail gel polish and nail polish. Compared with nail polish, there are advantages of nail gel polish. The components are as following: aliphatic urethane acrylate oligomer 10˜50(W %), alcohol soluble thermoplastics resin 10˜20(W %), acrylate monomer 15˜30(W %), photo initiator 1˜10(W %), pigment 1˜5(W %), dispersant 0.1˜2(W %), thickening agent 1˜5(W %), levelling agent 0.1˜1(W %), defomer 0.1˜0.5(W %). The nail gel polish refers to the invention abandons traditional nail polish remove way, and adopt remove way by medical alcohol. The medical alcohol, mainly use as sanitizer, is manufactured from plant material without methanol and formaldehyde. The medical alcohol is much safer and reduces hurt on nails and skin around nails of customers and nail technician while removing nail polish.

### CROSS-REFERENCE TO RELATED APPLICATIONS

The present application claims the benefit of Chinese Patent Application No. 201510867377.1 filed on Dec. 1, 2015, the contents of which are hereby incorporated by reference.

### FIELD OF THE INVENTION

This invention relates to the field of daily chemical products. The specific field refers to a nail gel polish and its manufacturing method.

### BACKGROUND OF THE INVENTION

The nail coatings used in human finger mainly are nail gel polish and nail polish. Compared with nail polish, there are advantages of nail gel polish as follow: the shorter time of forming film, the longer time holding on fingers, etc.

When want to change color in nails, we need to remove former color first. In general there are two popular removing methods, one is mechanic sanding, the other is using removing solvent. The former removing method, you need skilled craft and a professional device, if you are not skilled at sanding nail gel, you may make nails thinner and more brittle than before, what is worse customer may feel pain after service. If using removing solvent, because of the main components of removing solvent are acetone, ethyl acetate or butyl acetate, with pungent odor the liquid may hurt respiratory passage and skin of nail technicians and customer.

### SUMMARY OF THE INVENTION

The technical problem to be solved in the invention is to overcome the technical difficulties in removing nail gel polish. For exam that, it is hard to remove, the remove solvent is with high toxicity. Thus we invent a kind of nail gel polish which can be easily removed by alcohol.

### DETAILED DESCRIPTION OF ILLUSTRATED EMBODIMENTS

According to the invention, the nail gel polish which can be removed by alcohol can be successfully prepared, when added pigment color nail gel polish is prepared.

The special nail gel polish can be comprised of not only a wide variety of compounds containing one or more radical polymerizable unsaturated double bond but also thermoplastic alcohol soluble resin. Specific but not limited examples of alcohol soluble thermoplastic including the alcohol soluble thermoplastic resin which including the molecular structure is “—CH2-CH—CO—NH—CH2-O—CH2-CH—CH2-” , the molecular structure is

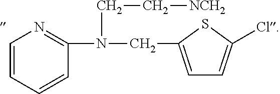

These aliphatic polyurethane resin and alcohol soluble thermoplastic resin are available from RAHN AG, SARTOMER CHINA, Rohm-Hass Co, Cytec Industries Inc, The DOW Chemical Company, Eastman Chemical Company.

The special nail gel polish can be comprised of monomer containing one or two unsaturated double bond. Typical examples include mono-functional group acrylate ester and bi-functional group acrylate ester. These monomer are available from The KAITEKI Company, Eternal Materials Co., Ltd.

The special nail gel polish contain a photo initiators at least one of these photo initiators which are 2-Hydroxy-2-methylpropiophenone, 1-hydroxy-cyclohexylphenyl-ketone,4,6-trimethylbenzoyldiphenyl-phosphine oxide or bis(2,6-difluoro-3-(1-hydropyrrol-1-yl)phenyl)titanocene, And mixtures thereof.

The special nail gel polish contain thickening agent is at least one of these thickening agents which are fumed silica, bentonite, modified castor oil or polyamide wax.

The special nail gel polish contain levelling agent is at least one of these levelling agents which is polyether modified silicone fluid levelling or polysiloxane-polyether copolymer.

The special nail gel polish contain defomer is at least one of these defomer which is polysiloxane emulsion, polymer-type without organic silicon or organic modified polysiloxane.

The special nail gel polish contain wetting agent is at least one of these wetting agent which is polyether silicone or containing pigment affinity group copolymers.

The special nail gel polish contain dispersant is at least one of containing pigment affinity group copolymers.

The pigment which can be incorporated into the concentrate include carbon black, D&C Black No. 2, D&C Black No. 3., FD&C Blue No. 1, D&C Blue No. 4, D&C Brown No. 1, FD&C Green No. 3, D&C Green No. 5, D&C Green No. 6, D&C Green No. 8, D&C Orange No. 4, D&C Orange No. 5, D&C Orange No. 10, D&C Orange No. 11, FD&C Red No. 4., D&C Red No. 6, D&C Red No. 7, D&C Red No. 17, D&C Red No. 21, D&C Red No. 22, D&C Red No. 27, D&C Red No. 28, D&C Red No. 30. D&C Red No. 31, D&C Red No. 33,

D&C Red No. 34, D&C Red No. 36, FD&C Red No. 40, D&C Violet No. 2, Ext. D&C Violet No. 2, FD&C Yellow No. 5, FD&C Yellow No. 6, D&C Yellow No. 7, Ext. D&C Yellow No. 7, D&C Yellow No. 8, D&C Yellow No. 10, D&C Yellow No. 11, titanium dioxide as well as others listed on the FDA color additives website, and Annex IV of Regulation(EC) No 1223/2009 of the European Parliament and of the Council of 30 Nov. 2009 on cosmetic prouducts, Coloring Agents Permitted in Cosmetics.

There are many possible embodiments of the special nail gel polish. In some embodiments the special nail gel polish is comprised of 10˜50% by weight aliphatic acrylate polyurethane, 10˜20% by weight alcohol soluble thermoplastic resin, 15˜30% by weight acrylate monomer, 1˜10% by weight photo initiator, 1˜5% by weight pigment, 0.1˜2% dispersant, 1˜5% by weight thickening agent, 0.1˜1% levelling agent, 0.1˜0.5% by weight defomer agent, 0.1˜0.3% by weight wetting agent.

The manufacturing method is as follow:

(1) Mixing and dissolving photo initiators and acrylate monomers under 1000 rpm˜2000 rpm for 15˜20 min in reaction kettle to dissolve uniformly.

(2) Keeping stirring condition, adding in turn aliphatic acrylate polyurethane, alcohol soluble thermoplastic resin, dispersant then mixing and dissolving under 1000 rpm-2000 rpm for 20-25 min in reaction kettle. Adding in turn wetting agents and levelling agents, mixing and dissolving under 1000 rpm-2000 rpm for 20-25 min in reaction kettle.

(3) Adding in turn pigment, defomer and mixing and dissolving, then put them in sand mill to grind until pigment particle size is less than 20 microns. The nail gel polish is obtained.

As to existing technologies the invention has advantages as follow: the nail gel polish refer to the invention abandons traditional nail polish remove way, and adopt remove way by medical alcohol. The medical alcohol, mainly use as sanitizer, is manufactured from plant material without methanol and formaldehyde. The medical alcohol is much safer and reduces hurt on nails and skin around nails of customers and nail technicians while removing nail polish.

### EXAMPLES

### Example 1

Aliphatic acrylate polyurethane 28% by weight

Alcohol soluble thermoplastic resin with “—CH2-CH—CO—NH—CH2-O—CH2-CH—CH2-” structure 17% by weight

Mono-functional acrylate monomer 15% by weight

Bi-functional acrylate monomer 8% by weight

2,4,6-trimethylbenzoyldiphenyl-phosphine oxide 4.6% by weight

1-hydroxy-cyclohexylphenyl-ketone 2% by weight

Bentonite 5% by weight

Polymer-type defomer without organic silicon 0.3% by weight

Polyether modified silicone fluid levelling agent 0.2% by weight

Polyether silicone wetting agent 0.3% by weight

Red pigment 3% by weight

Containing pigment affinity group copolymers 0.6% by weight

### Example 2

Aliphatic acrylate polyurethane 15% by weight

Alcohol soluble thermoplastic with “—CH2-CH—CO—NH—CH2-O—CH2-CH—CH2—”, structure 20% by weight

Mono-functional acrylate monomer 15% by weight

2,4,6-trimethylbenzoyldiphenyl-phosphine oxide 10% by weight

Bentonite 5% by weight

Polymer-type defomer without organic silicon 0.1% by weight

Polyether modified silicone fluid levelling agent 1% by weight

Polyether silicone wetting agent 0.3% by weight

Red pigment 5% by weight

Containing pigment affinity group copolymers 0.1% by weight

### Example 3

Aliphatic acrylate polyurethane 50% by weight

Alcohol soluble thermoplastic with

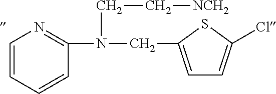

structure 10 % by weight

Mono-functional acrylate monomer 10% by weight

Bi-functional acrylate monomer 30% by weight

1-hydroxy-cyclohexylphenyl-ketone 1% by weight

Bentonite 1% by weight

Polymer-type defomer agent without organic silicon 0.5% by weight

Polyether modified silicone fluid levelling agent 0.1% by weight

Polyether silicone wetting agent 0.3% by weight

Red pigment 1% by weight

Containing pigment affinity group copolymers 0.5% by weight

Disclose the manufacturing method of the invention as follow:

(1) Mixing and dissolving2,4,6-trimethylbenzoyldiphenyl-phosphine oxide, 1-hydroxy-cyclohexylphenyl-ketone, Mono-functional acrylate monomer and Bi-functional acrylate monomer under 1000 rpm˜2000 rpm for 15˜20 min in reaction kettle to dissolve uniformly.

(2) Keeping stirring condition, adding in turn aliphatic acrylate polyurethane, alcohol soluble thermoplastic with “—CH2-CH—CO—NH—CH2-O —CH2-CH—CH2—” structure, containing pigment affinity group copolymers then mixing and dissolving under 1000 rpm˜2000 rpm for 20˜25 min in reaction kettle. Adding in turn polyether silicone wetting agent and polyether modified silicone fluid levelling agent, mixing and dissolving under 1000 rpm˜2000 rpm for 20˜25 min in reaction kettle.

(3) Adding in turn red pigment, polymer-type defomer without organic silicon mixing and dissolving, then put them in sand mill to grind for 30 minutes until pigment particle size is less than 20 microns. The nail gel polish is obtained.

Compare

Compared Example 1 nail gel polish with existing nail gel polish, both applied to the nail, after cured by UV light, using 75% alcohol to remove, observation as table 1

TABLE 1

| Example 1 nail gel polish | Nail gel polish in market |
|---|---|
| After 10 min, nail gel polish is crack | After 10 min, nail gel is hard to remove, |
| and can be easily removed, little | only with professional tool to push that |
| hurt on finger nail, | nail gel can be removed. Thus, finger nail |
| | is hurt. |

The present invention, therefore, is well adapted to carry out the objects and attain the ends and advantages mentioned, as well as others inherent therein. While the invention has been depicted and described and is defined by reference to particular preferred embodiments of the invention, such references do not imply a limitation on the invention, and no such limitation is to be inferred. The invention is capable of considerable modification, alteration and equivalents in form and function, as will occur to those ordinarily skilled in the pertinent arts. The depicted and described preferred embodiments of the invention are exemplary only and are not exhaustive of the scope of the invention. Consequently, the invention is intended to be limited only by the spirit and scope of the appended claims, giving full cognizance to equivalents in all respects.

What is claimed is:

1. A nail gel polish, comprising the following components:
aliphatic urethane acrylate oligomer 15%-50% by weight;
alcohol soluble thermoplastic resin 10%-20% by weight;
acrylate monomer 15%-30% by weight;
photoinitiator 1%-10% by weight;
pigment 1%-5% by weight;
dispersant 0.1%-2% by weight;
thickening agent 1%-5% by weight;
levelling agent 0.1%4% by weight;
defomer 0.1%0.5% by weight.

2. The nail gel polish according to claim 1, wherein the contained alcohol soluble thermoplastic resin must be one of these two alcohol soluble thermoplastics which contain these molecule structures as follow: “CH2-CH—CO—NH—CH2-O—CH2-CH—CH238 or

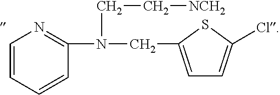

3. The nail gel polish according to claim 1, wherein the contained acrylate monomer must be one of these monomers which are mono-functional acrylate monomer and bi-functional acrylate monomer.

4. The nail gel polish according to claim 1, wherein the contained photo initiators must be one of these photo initiators which are 2-Hydroxy-2-methylpropiophenone, 1-hydroxy-cyclohexylphenyl-ketone, 2,4,6-trimethylbenzoyldiphenyl-phosphine oxide or bis(2,6difluoro-3-(1-hydropyrrol-1-yl)phenyl)titanocene, or mixtures of those above.

5. The nail gel polish according to claim 1, wherein the contained thickening agents must be one of these thickening agents which are fumed silica, bentonite, modified castor oil, polyamide wax, or mixtures of those above.

6. The nail gel polish according to claim 1, wherein the contained levelling agents must be one of polyether modified silicone, polysiloxane-polyether copolymer, or mixtures of those above.

7. The nail gel polish according to claim 1, wherein the contained defoamers must be one of polysiloxane emulsion, polymer-type defoamer without organic silicon, organic modified polysiloxane, or mixtures of those above.

8. The nail gel polish according to claim 1, wherein the contained dispersant must be containing pigment affinity group copolymers.

9. The nail gel polish according to claim 1, wherein the contained wetting agent must be one of polyether silicone, pigment affinity group copolymer, or mixture of those above.

10. A method of manufacturing the nail gel polish according to claim 1, wherein said manufacturing method comprising following steps:
(1) mixing and dissolving photo initiators and acrylate monomers under 1000 rpm˜2000 rpm for 15˜20 min in reaction kettle by high speed dispersion machine;
(2) keeping stirring condition, adding aliphatic urethane acrylate oligomer, alcohol soluble thermoplastic resin and dispersant sequentially then mixing and dissolving under 1000 rpm˜2000 rpm for 20˜25 min in reaction kettle by high speed dispersion machine, adding wetting and levelling agents, mixing and dissolving for 20˜25 min in reaction kettle by high speed dispersion machine to dissolving homogeneous; and
(3) adding pigment, defomer, mix and dissolve in reaction kettle by high speed dispersion machine, then put them into sand mill to grind until pigment particle size is less than 20 microns, so as to obtain nail gel polish.
